(19) United States
(12) Patent Application Publication
(10) Pub. No.: US 2025/0195913 A1
(43) Pub. Date: Jun. 19, 2025

(54) LIGHT THERAPY DEVICE STAND

(71) Applicant: Mark Sawyer, Lake Mary, FL (US)

(72) Inventor: Mark Sawyer, Lake Mary, FL (US)

(21) Appl. No.: 18/971,940

(22) Filed: Dec. 6, 2024

Related U.S. Application Data
(63) Continuation of application No. 18/643,452, filed on Apr. 23, 2024.
(60) Provisional application No. 63/612,247, filed on Dec. 19, 2023.
(60) Provisional application No. 63/612,237, filed on Dec. 19, 2023.

(51) Int. Cl. A61N 5/06

(52) U.S. Cl. CPC A61N 5/0613; A61N 5/0616; A61N 5/0617; A61N 5/0618; A61N 2005/0626; A61N 2005/0652; A61N 2005/0659; A61N 2005/0663

(57) ABSTRACT

A light therapy device stand includes a table mount having a clamp and a pair of inverted brackets for selectively engaging a table. A vertical extension arm extends upward from the table mount, and a horizontal extension arm rotates 360 degrees along a horizontal plane. An adjustable arm assembly extends from the distal end of the horizontal extension arm and pivots between a fully raised and fully lowered position. A panel attachment assembly is secured along the distal end of the adjustable arm assembly and is connected to a light therapy device. The adjustable arm assembly maintains the mounting assembly in a horizontal orientation regardless of the position of the parallel arms, and the mounting assembly moves the light therapy device in a plurality of different horizontal and vertical orientations.

### CROSS-REFERENCE TO RELATED APPLICATIONS

This application claims the benefit of U.S. Application Ser. No. 63/612,247 filed on Dec. 19, 2023, and U.S. patent application Ser. No. 18/643,452 filed on Apr. 23, 2024, the contents of each of which are incorporated herein by reference.

### TECHNICAL FIELD

The present invention relates generally to the field of light therapy devices, and more particularly to a stand for positioning one or more light therapy devices onto a desk or other fixed structure in a specified orientation.

### BACKGROUND

The statements in this section merely provide background information related to the present disclosure and may not constitute prior art.

Scientific research has shown that exposing human skin to certain types of light can have a meaningful effect on the person's overall health. Depending on the intensity and spectrum/wavelength of the light, these effects can include reducing stress, eliminating headaches, promoting hair growth, increasing collagen production, reducing the appearance of acne, and even fat reduction.

Many commercially available light therapy devices are manufactured to include an industry standard height/length of about 19 inches and a width of about 8-10 inches so as to be suitable for providing light therapy to a single area of a user's body such as the head, chest, or back, for example. In this regard, it is important that a user be able to accurately position the device to ensure the desired portion of their body receives the treatment.

Most commercially available light therapy devices are provided with a mounting kit having a pair of posts that are screwed into the top surface of the device which engage a pair of elongated cords and a pulley system to allow the device to be suspended from a door or wall via a hook. Unfortunately, these devices are cumbersome to use, and do not provide the ability for a user to easily maneuver the light therapy device to direct the light emissions at different orientations.

Additionally, these kits do not allow a user to rotate a light therapy device so as to be positioned in either a horizontal or vertical manner, so as to change the width/height of the light output. Additionally, these kits are not easily used by individuals suffering from limited mobility who need to remain sitting during a treatment, and/or users who wish to receive a treatment on their arms or hands without resting the same on a desk or table.

The present invention, directed to a light therapy device stand differs from the conventional art in several aspects. The manner by which will become more apparent in the description which follows, particularly when read in conjunction with the accompanying drawings.

### SUMMARY OF THE INVENTION

The present invention is directed to a light therapy device stand. One embodiment of the present invention can include a table mount having a clamp and a pair of inverted brackets for selectively engaging a table, desk, or other horizontal structure. A vertical extension arm can extend upward from the table mount, and a horizontal extension arm can be secured to the vertical arm in a manner that permits the horizontal arm to rotate 360 degrees along a horizontal plane. An adjustable arm assembly can extend from the distal end of the horizontal extension arm. The adjustable arm assembly can include two parallel members that pivot between a fully raised and fully lowered position, and a panel attachment assembly can be secured along the distal end of the adjustable arm assembly.

In one embodiment, a height of the horizontal extension arm is adjustable via a locking collar, and the adjustable arm assembly can transition about 60 degrees above and below the horizontal plane in the raised and lowered position. In one embodiment, a spring is provided with the adjustable arm assembly to bias the two parallel arms in the fully raised position. The adjustable arm assembly can also function to maintain the mounting assembly in a horizontal orientation regardless of the position of the parallel arms.

In one embodiment, the stand can move a connected light therapy device in a plurality of different horizontal and vertical directions, along with rotating the device in any vertical and horizontal orientations to allow the light output to reach a user in any position.

This summary is provided merely to introduce certain concepts and not to identify key or essential features of the claimed subject matter.

### BRIEF DESCRIPTION OF THE DRAWINGS

Presently preferred embodiments are shown in the drawings. It should be appreciated, however, that the invention is not limited to the precise arrangements and instrumentalities shown.

FIG. 1 is an exploded parts view of a light therapy device stand that is useful for understanding the inventive concepts disclosed herein.

FIG. 2A is a perspective view of the table mount of the light therapy device stand, in accordance with one embodiment of the invention.

FIG. 2B is another perspective view of the table mount of the light therapy device stand, in accordance with one embodiment of the invention.

FIG. 3 is a perspective view of the table mount, the vertical extension arm and the horizontal extension arm of the light therapy device, in accordance with one embodiment of the invention.

FIG. 4 is a cutout view of the horizontal extension arm of the light therapy device stand, in accordance with one embodiment of the invention.

FIG. 5 is a cutout view of the adjustable extension arm assembly of the light therapy device stand, in accordance with one embodiment of the invention.

FIG. 6A is a cutout view of the panel attachment assembly of the light therapy device stand, in accordance with one embodiment of the invention.

FIG. 6B is a partial cutout view of the adjustable extension arm assembly and the panel attachment assembly of the light therapy device stand, in accordance with one embodiment of the invention.

FIG. 6C is a partial cutout view of the adjustable extension arm assembly and the panel attachment assembly of the light therapy device stand, in accordance with one embodiment of the invention.

FIG. 7 is a side view of the light therapy device stand in operation, in accordance with one embodiment of the invention.

FIG. 8A is a perspective view of the light therapy device stand in operation with a light therapy device, in accordance with one embodiment of the invention.

FIG. 8B is a side view of the light therapy device stand in operation with a light therapy device, in accordance with one embodiment of the invention.

FIG. 8C is another perspective view of the light therapy device stand in operation with a light therapy device, in accordance with one embodiment of the invention.

FIG. 8D is another perspective view of the light therapy device stand in operation with a light therapy device, in accordance with one embodiment of the invention.

### DETAILED DESCRIPTION OF THE INVENTION

While the specification concludes with claims defining the features of the invention that are regarded as novel, it is believed that the invention will be better understood from a consideration of the description in conjunction with the drawings. As required, detailed embodiments of the present invention are disclosed herein; however, it is to be understood that the disclosed embodiments are merely exemplary of the invention which can be embodied in various forms. Therefore, specific structural and functional details disclosed herein are not to be interpreted as limiting, but merely as a basis for the claims and as a representative basis for teaching one skilled in the art to variously employ the inventive arrangements in virtually any appropriately detailed structure. Further, the terms and phrases used herein are not intended to be limiting but rather to provide an understandable description of the invention.

### Definitions

As described herein, a “unit” and “section” mean a series of identified physical components which are linked together and/or function together to perform a specified function.

As described throughout this document, the term “about” “approximately” “substantially” and “generally” shall be used interchangeably to describe a feature, shape, or measurement of a component within a tolerance such as, for example, manufacturing tolerances, measurement tolerances or the like.

As described herein, the term “removably secured,” and derivatives thereof shall be used to describe a situation wherein two or more objects are joined together in a non-permanent manner so as to allow the same objects to be repeatedly joined and separated.

As described throughout this document, the term “complementary shape,” and “complementary dimension,” shall be used to describe a shape and size of a component that is identical to, or substantially identical to the shape and size of another identified component within a tolerance such as, for example, manufacturing tolerances, measurement tolerances or the like.

As described herein, the term “slidingly engage”, “telescopically connected” and derivatives thereof shall be used interchangeably to describe a situation wherein two or more identified objects are connected linearly such that one identified part slides into and out from the other identified part so as to selectively lengthen or shorten the total linear length of the assembly.

As described herein, the term “connector” includes any number of different elements that work alone or together to join two items together in a nonpermanent manner. Several nonlimiting examples of connectors include, but are not limited to, thread-to-connect, twist-to-connect, and push-to-connect type devices, opposing strips of hook and loop material (e.g., Velcro®), attractively oriented magnetic elements or magnetic and metallic elements, buckles, clamps, sockets, clips, and compression fittings, among others, for example.

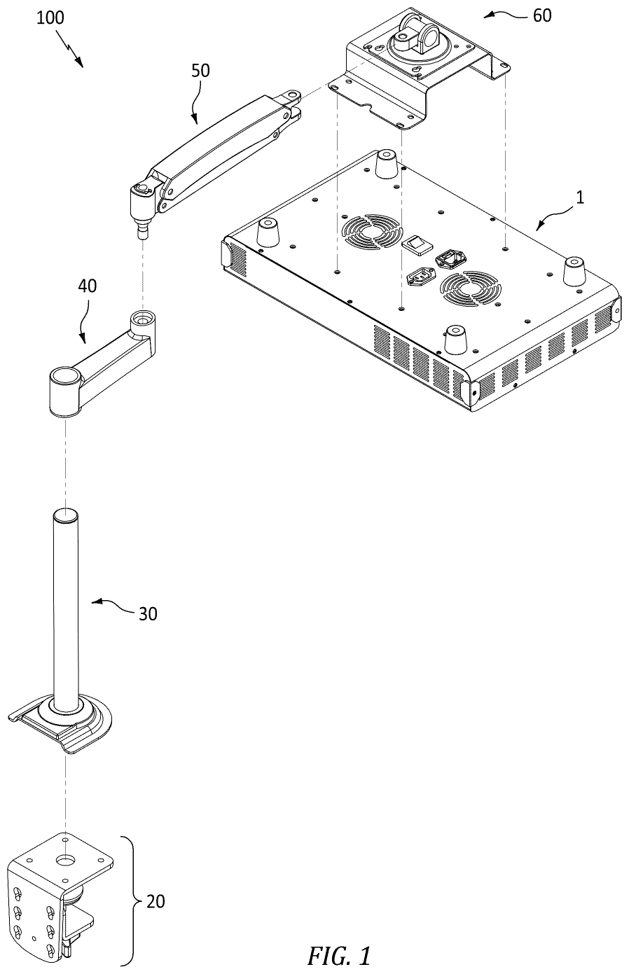

FIGS. 1-8D illustrate one embodiment of a light therapy device stand 10 that are useful for understanding the inventive concepts disclosed herein. In each of the drawings, identical reference numerals are used for like elements of the invention or elements of like function. For the sake of clarity, only those reference numerals are shown in the individual figures which are necessary for the description of the respective figure. For purposes of this description, the terms “upper,” “bottom,” “right,” “left,” “front,” “vertical,” “horizontal,” and derivatives thereof shall relate to the invention as oriented in FIG. 1.

As described herein, the inventive stand 10 can function to engage and position one or more light therapy devices such as those described in copending U.S. patent application Ser. No. 18/643,452 to Sawyer, the contents of which are incorporated herein by reference. However, other types of light therapy devices may also be used.

As shown in the drawings, one embodiment of the light therapy device stand 10 for engaging and positioning a light therapy device 1 can include, essentially, a table mount 20, having a flanged vertical extension arm 30, a rotating horizontal extension arm 40, an adjustable extension arm assembly 50, and a panel attachment assembly 60.

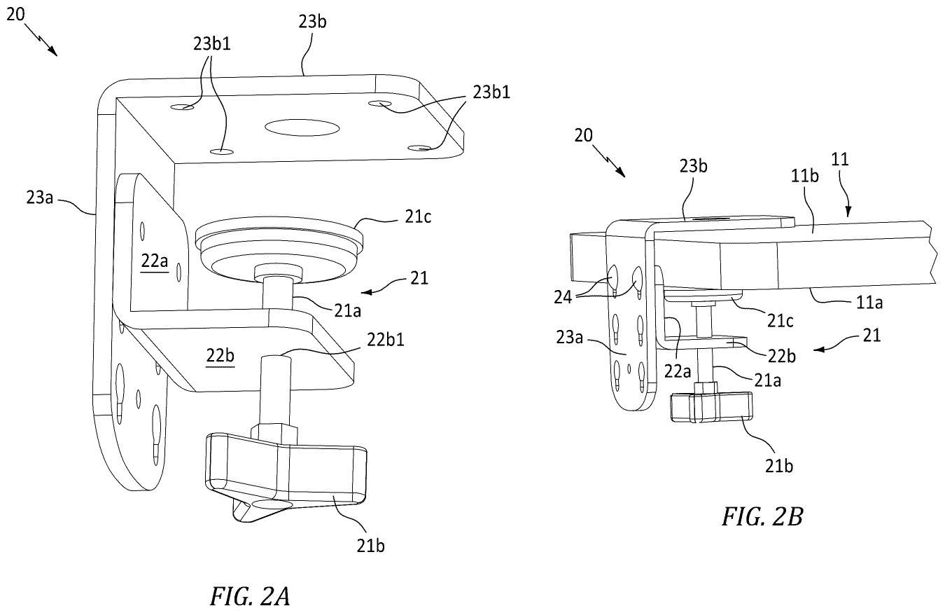

The table mount 20 can include any number of devices or individual components capable of engaging a flat horizontal structure, such as a desk, counter or tabletop, for example. As shown best at FIGS. 2A and 2B, one embodiment of the mount 20 can include a clamp 21 having an adjusting screw 21a and a knob 21b that is selectively tightened to secure a swivel head 21c onto the bottom surface 11a of a table 11 or other structure. In one embodiment, a first L-shaped bracket is positioned such that the first half of the bracket 22a is positioned vertically, so as to be parallel to the clamp 21, and the second half of the bracket 22b is positioned horizontally, so as to be perpendicular to the clamp. When so positioned, the adjusting screw 21a extends through a central opening 22b1 on the horizontal portion of the bracket in order to permit vertical movement of the first bracket.

In one embodiment, a second L-shaped bracket is positioned adjacent to the first bracket. As shown, the second bracket is positioned such that a first half of the second bracket 23a is positioned vertically and is secured against the vertical half of the first bracket 22a, via connectors 24 such as screws or bolts, for example. When so positioned, the second half of the bracket 23b will be located horizontally above and parallel to the second half of the first bracket 22b. As such, the second half of the bracket 23b will rest against the top surface 11b of the table 11 when the clamp 21 is tightened. This arrangement of the clamp and brackets functioning to securely engage the table, desk, or other flat surface to support the stand 10 during use with a light therapy device 1 as described below.

Although described with regard to a clamp that is selectively tightened via a knob, this is for illustrative purposes only, as any type of connector and/or bracket assembly capable of removably engaging a table or other surface to support the device components in the manner described below are also contemplated.

As described herein, the table mount 20 may be formed from materials that are, for example, relatively strong and stiff for their weight. Several nonlimiting examples include but are not limited to various metals or metal alloys (e.g., aluminum, steel, titanium, or alloys thereof), plastic/polymers (e.g., high-density polyethylene (HDPE), rigid polyvinyl chloride (PVC), malleable polyethylene terephthalate (PET)), and/or various composite materials.

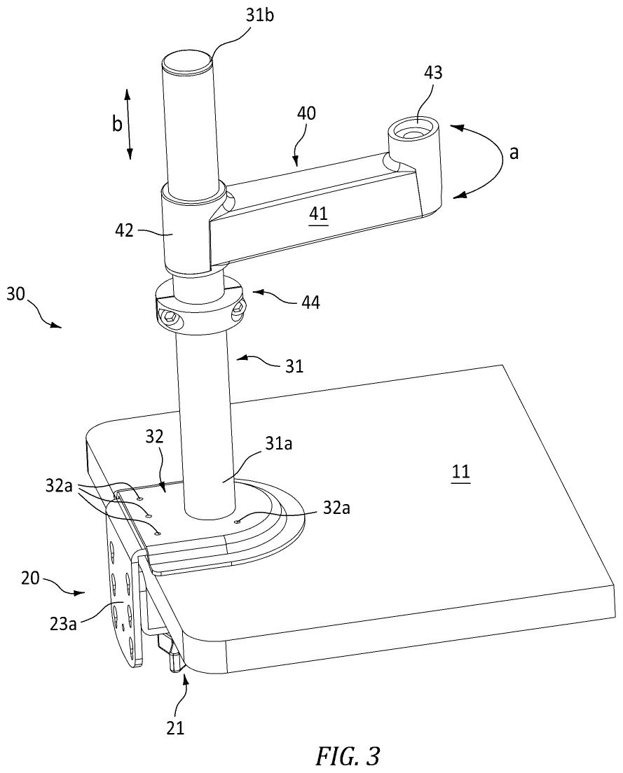

FIG. 3 illustrates one embodiment of the vertical extension arm 30 that extends upward from the table mount 20 when the table mount is secured onto a horizontal surface 11. In one embodiment, the vertical extension arm 30 can include an elongated tubular member 31 having a bottom end 31a, a top end 31b, and a flange 32 along the bottom end. The flange can include a plurality of threaded apertures 32a that are positioned at complementary locations to openings 23b1 located on the second bracket 23. In this regard, additional connectors such as bolts, for example, can pass through the openings to engage the threaded apertures to secure the flange 32 to the top half of the bracket. When so positioned the tubular member 31 will be positioned vertically so as to extend upward from the table 11.

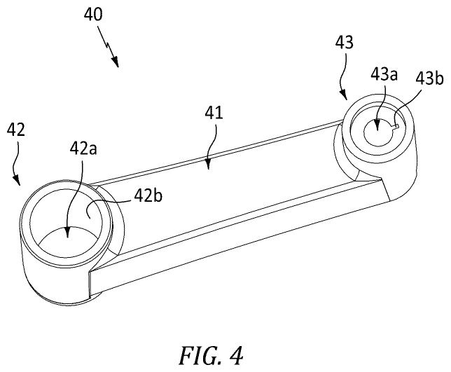

The horizontal extension arm 40 can be connected to the vertical extension arm 30 and can extend outward therefrom in a horizontal orientation. As shown best at FIGS. 3 and 4, one embodiment of the arm 40 can include an elongated member 41 having a rotating joint 42 along the first end, and a receptacle 43 along the second end.

The rotating joint 42 can include a central opening 42a that is shaped and sized to receive the second end 31b of the extension arm member 31. As described herein, the joint 42 can include a tapered roller bearing 42b or other such mechanism that is capable of rotating the horizontal arm 360 degrees about the vertical arm 30 along a single axis as shown by arrow a. One example of a suitable rotating joint for use herein includes model 5709k88 tapered roller

bearing joint that are commercially available from McMaster-Carr®. Of course, any number of other devices are also contemplated.

The receptacle 43 can function to receive and engage the coupler of the below described adjustable arm assembly 50. In one embodiment, the receptacle can include a central opening 43a having a slot 43b or channel along one side. The opening and slot including a shape and size that is suitable for receiving and engaging the protrusion of the coupler 54 described below.

In one embodiment, a collar clamp 44 is positioned along the vertical arm 31 at a location beneath the rotating joint 42. In this regard, the horizontal arm 40 can move vertically along the height of the vertical arm (see arrow b) and can be secured in the desired position via the collar clamp 44. Of course, any number of other types of devices capable of securing the horizontal arm onto the vertical arm in a manner that permits the arms to move/rotate about each other and to be positioned at varying locations along the height of the vertical arm are also contemplated.

As described herein, each of the extension arms 30 and 40 can include any number of different lengths and/or cross-sectional thicknesses for accommodating the forces applied to the system during use. Additionally, each of the extension arms may be formed from materials that are, for example, relatively strong and stiff for their weight. Several nonlimiting examples include, but are not limited to various metals or metal alloys (e.g., aluminum, steel, titanium, or alloys thereof), plastic/polymers (e.g., high-density polyethylene (HDPE) or polyethylene terephthalate (PET)), and/or various composite materials (e.g., carbon fibers in a polymer matrix, etc.).

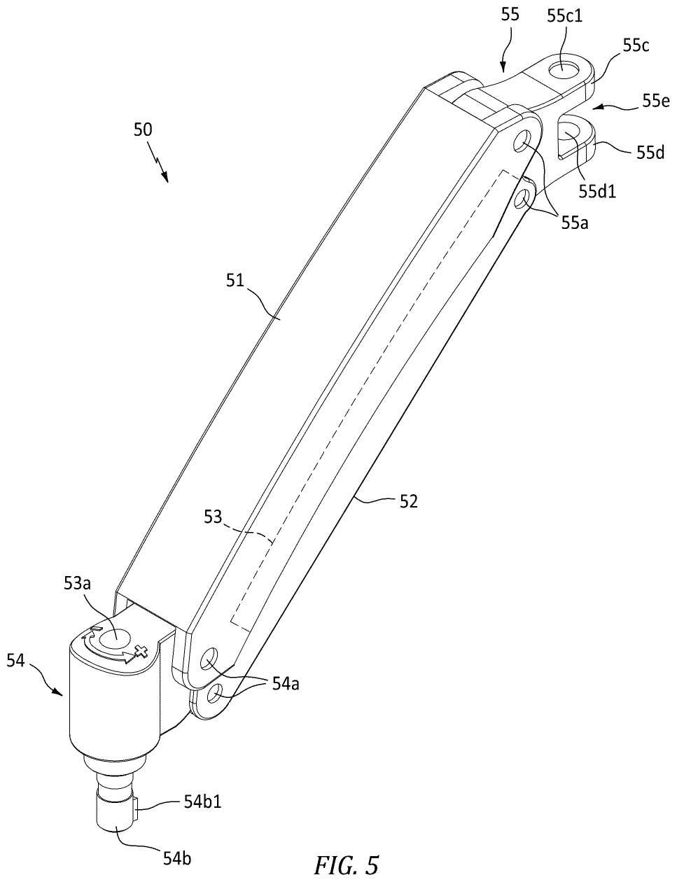

The adjustable arm assembly 50 can extend outward from the distal end of the horizontal arm 40 and can position the mounting assembly 60 in a plurality of different orientations. As shown best at FIG. 5, one embodiment, the adjustable arm assembly 50 can include two elongated arms 51 and 52 having a first coupler 54 along their proximal ends, and a second coupler 55 along their distal ends.

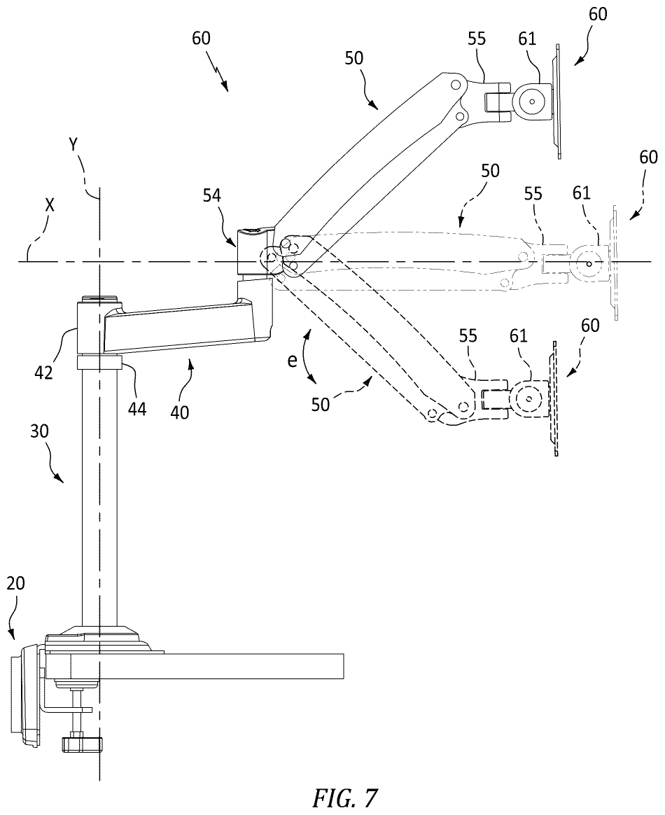

In one embodiment, a spring 53 is connected between the first and second couplers 54 and 55, and is routed between the arms 51 and 52. The spring serves to bias the adjustable arm assembly in the fully raised position (See FIG. 7), and can include a tension screw 53a or other known tensioning mechanism to allow a user to adjust the spring tension to increase or decrease the force required to move the arms up and down.

In one embodiment, the first coupler 54 is connected to the arms 51 and 52 via a pair of shafts 54a that extend perpendicularly through the coupler and arms. The shafts can be aligned vertically to permit the arms 51 and 52 to pivot up and down relative to the first coupler 54 as shown below at FIG. 7.

In one embodiment, the first coupler can include a male fitting 54b that extends downward from the bottom end of the coupler. The fitting can be removably positioned within the receptacle 43 and will include a shape and a size that is complementary to the shape of the receptacle opening 43a. In various embodiments, the fitting permits the arm assembly 50 to pivot, rotate or otherwise move horizontally relative to the horizontal arm 40.

In one embodiment, the fitting can include a lip 54b1 or ridge, for example, that extends outward therefrom. The lip can include a shape, size and location that is complementary to the channel 43b of the receptacle 43 so as to be positioned therein when the fitting is positioned within the opening 43a. Such a feature functioning to prevent horizontal rotation/movement of the arm assembly 50 relative to the arm 40.

The second coupler 55 functions to connect the adjustable arm assembly to the panel attachment assembly 60. In one embodiment, the second coupler 55 is also connected to the arms 51 and 52 via a pair of shafts 55a that extend perpendicularly through the coupler and arms. The shafts are also aligned vertically to permit the arms to pivot up and down as described above. As will be described below, this arrangement permits the second coupler to maintain the panel attachment assembly in a purely horizontal position at all times, regardless of the position of the arm assembly 50.

As shown, the second coupler 55 can include two protruding surfaces 55c and 55d that are separated by a central opening 55e. Holes 55c1 and 55d1 extend vertically through the protruding surfaces to receive hardware for engaging the panel attachment assembly 60.

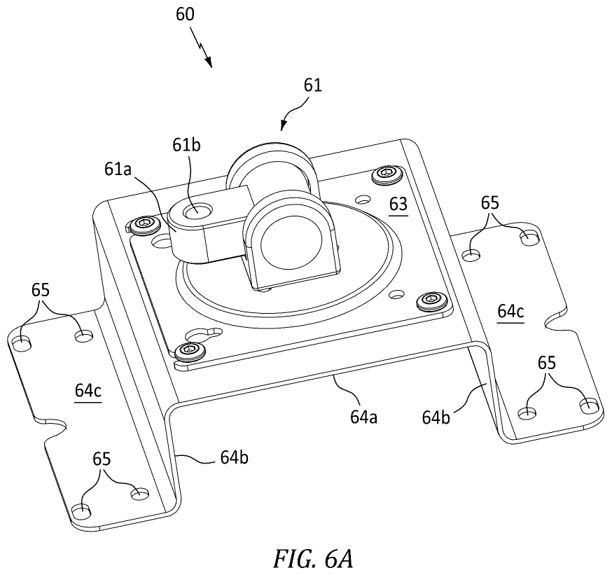

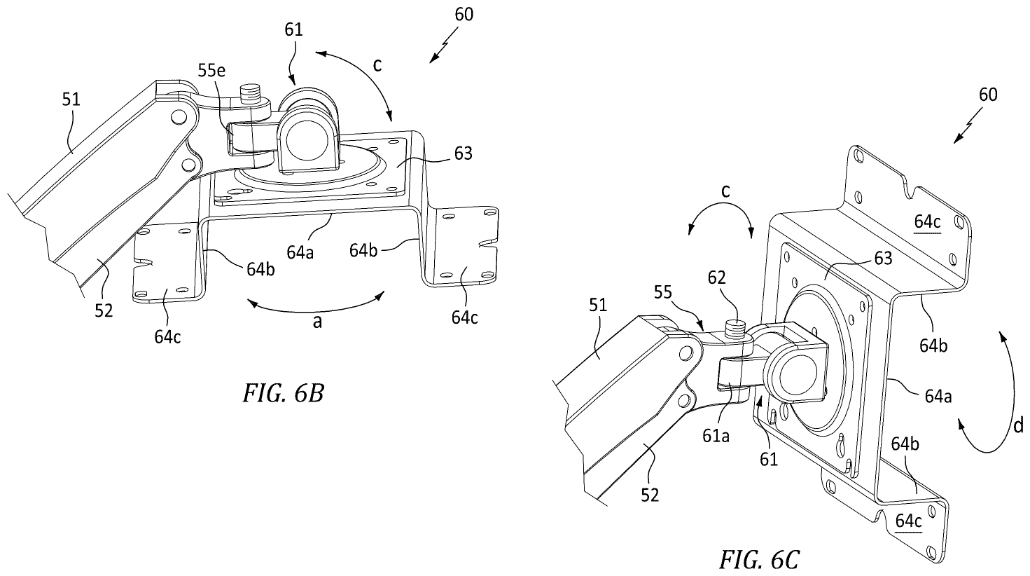

As shown in FIGS. 6A-6C, the panel attachment assembly 60 can include a swivel hinge 61 having a first end 61a that is positioned within the central opening 55e of the second coupler 55. When so positioned, holes 55c1 and 55d1 will align with hole 61b to receive mounting hardware such as a bolt 62, for example.

The bottom of the swivel hinge 61 is connected to a rotating flange 63 that is connected to a generally U-shaped mounting bracket. The combination of the hinge 61 and flange 63 permits the device to pivot, rotate and/or otherwise move the mounting bracket and an attached light therapy device both horizontally (see arrow c) and vertically (see arrow d) relative to the assembly 50.

In one embodiment, the bracket can include a planar central plate 64a, having two rails 64b extending orthogonally outward therefrom. A pair of mounting plates 64c extend orthogonally outward from the bracket rails so as to be parallel to the central plate. In one embodiment, a plurality of holes 65 are positioned along the mounting plates 64c. Each of the holes have a shape and a location that is complementary to the threaded apertures 2 on the back surface of a light therapy device 1.

As shown best at FIG. 7, the first coupler 54 can pivot, rotate and/or otherwise move (see arrow e) the arms of the adjustable arm assembly 50 about sixty degrees vertically, so as to position the panel attachment assembly anywhere between about thirty degrees above and thirty degrees below a horizontal plane X, (e.g., the fully raised position and the fully lowered position, respectively). Moreover, the internal spring connecting the first and second couplers together maintains the distal end of the second coupler 55 in a completely horizontal position at all times, regardless of the movement of the assembly 50. Such a feature is important to ensure that a light therapy device mounted onto the stand is positioned in a purely vertical position, a purely horizontal position, or a position customized by the user via manipulation of the swivel hinge 61 as the assembly 50 moves anywhere between the fully raised and fully lowered position shown at FIG. 7.

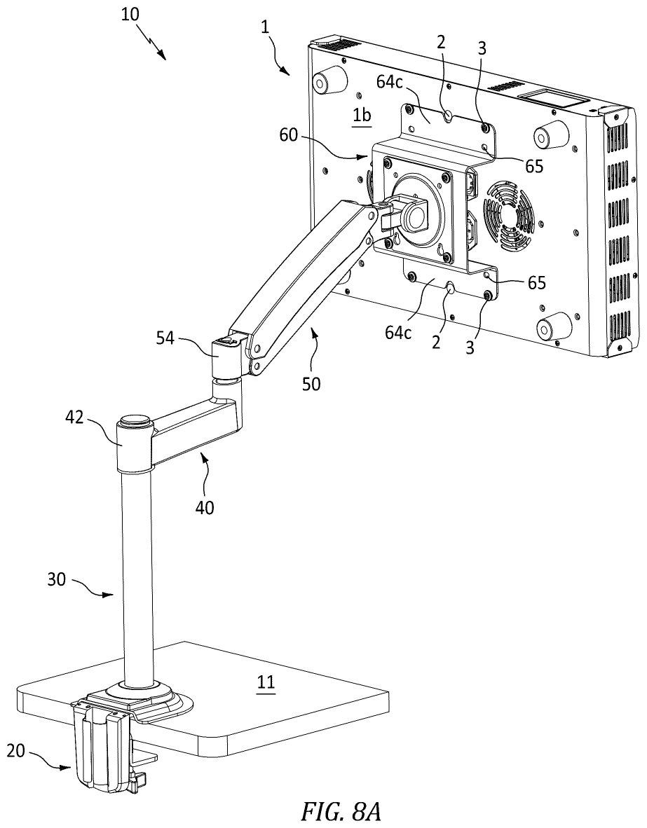

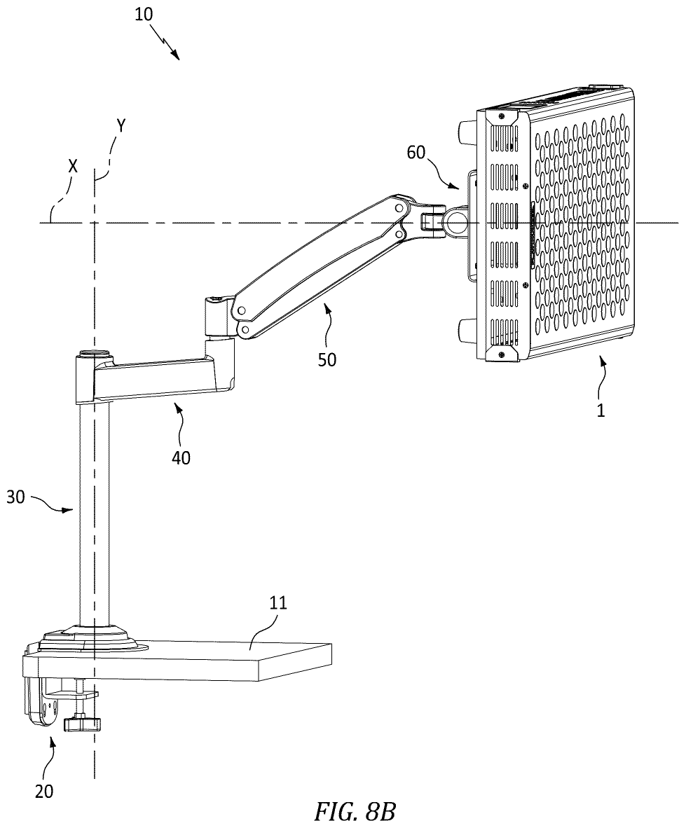

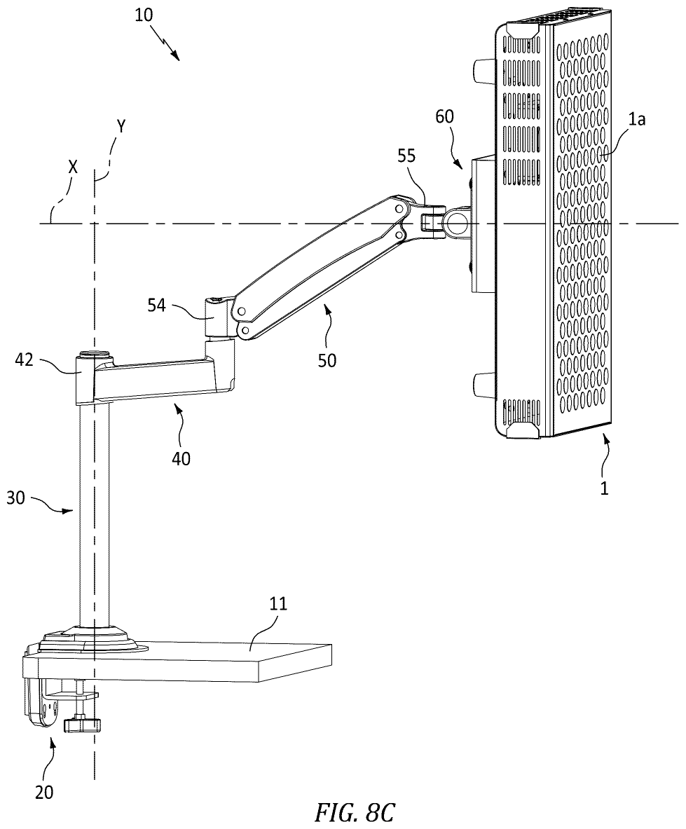

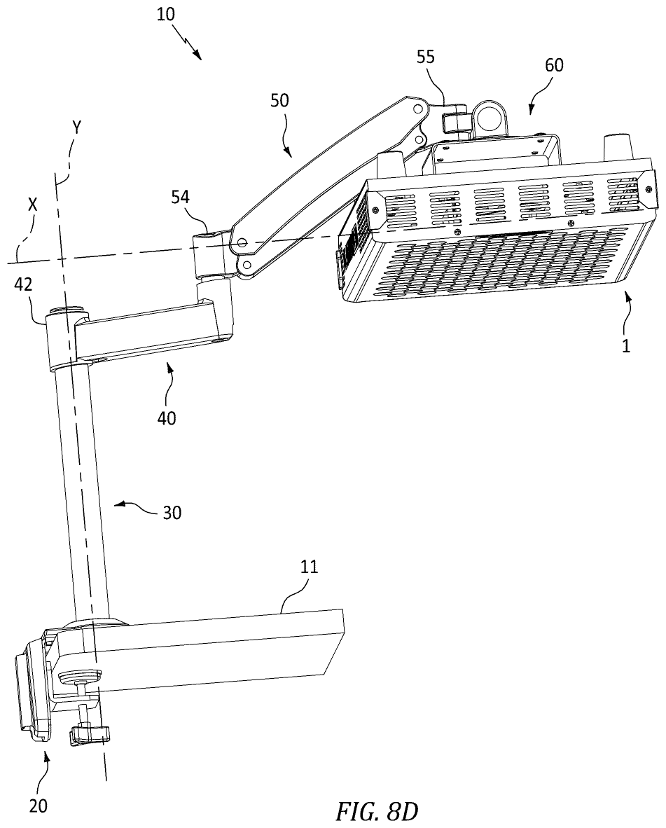

FIGS. 8A-8D illustrate one embodiment of the stand in operation. As shown, the back surface 1b of a light therapy device 1 can be positioned against the mounting plates 64c and secured to the stand 10 via bolts 3 that pass through the holes 65 to engage the mounting apertures 2 on the back surface 1b of the device. When so positioned, a user can adjust the orientation of the attached panel so that the front surface 1a emits light horizontally outward (along plane X) in a parallel direction to the table 11 when the device is positioned both horizontally-FIG. 8B, for a wide light distribution, or vertically-FIG. 8C, for a tall light distribution. Additionally, a user can adjust the orientation of the attached panel so that the front surface 1a emits light vertically downward (along plane Y) perpendicularly to the table 11 as shown at FIG. 8D.

In this regard, the inventive stand 10 can move or orient a connected light therapy device 1 in virtually any direction such as horizontally and vertically, the stand can pivot/rotate the device vertically up and down, and the stand can pivot/rotate the device horizontally along the horizontal plane.

The ability to securely position and orient a light therapy device anywhere between a purely horizontal and purely vertical position, along with being able to rotate the device itself anywhere between a vertical and horizontal orientation, including diagonally, is an important feature of the stand. Such a feature allows individuals with limited mobility to sit while receiving a light therapy treatment on their upper body and to adjust the orientation of the light therapy device. Moreover, by directing the light emissions vertically onto the same table to which the device is located, the stand advantageously allows users to receive light therapy treatments on their hands, arms or other body parts while resting the same on the table 11. Such a feature being critical for utilization by those with mobility issues, paralysis or other ailments who would not otherwise be able to suspend their arms in front of a device without assistance.

As to a further description of the manner and use of the present invention, the same should be apparent from the above description. Accordingly, no further discussion relating to the manner of usage and operation will be provided.

As described herein, one or more elements of the light therapy device stand 10 can be secured together utilizing any number of known attachment means such as, for example, screws, glue, compression fittings and welds, among others. Moreover, although the above embodiments have been described as including separate individual elements, the inventive concepts disclosed herein are not so limiting. To this end, one of skill in the art will recognize that one or more individually identified elements may be formed together as one or more continuous elements, either through manufacturing processes, such as welding, casting, or molding, or through the use of a singular piece of material milled or machined with the aforementioned components forming identifiable sections thereof.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Likewise, the term “consisting” shall be used to describe only those components identified. In each instance where a device comprises certain elements, it will inherently consist of each of those identified elements as well.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A light therapy device stand, comprising:
a table mount that is configured to engage a flat structure;
a first extension arm that is connected to the base;
a second extension arm that is connected to the first extension arm in an orthogonal manner;
an adjustable arm assembly that is connected to the second extension arm; and
a panel attachment assembly that is connected to a distal end of the adjustable arm assembly,
wherein the panel attachment assembly is configured to receive and engage a back surface of a light therapy device.

2. The stand of claim 1, wherein the base includes a clamp, and a bracket for engaging the flat structure.

3. The stand of claim 2, wherein the first extension arm includes a flange along a first end, and the flange is removably connected to the bracket.

4. The stand of claim 3, wherein the first extension arm is positioned horizontally when the base is secured onto the flat surface.

5. The stand of claim 1, wherein the second extension arm includes a rotating joint along the first end.

6. The stand of claim 5, wherein the second extension arm is configured to rotate 360 degrees relative to the first extension arm.

7. The stand of claim 1, wherein the adjustable arm assembly is connected to a second end of the second extension arm, and the adjustable arm assembly is configured to transition between a fully raised position and a fully lowered position.

8. The stand of claim 7, wherein the panel attachment assembly includes a bracket having a pair of mounting plates for engaging the back surface of the light therapy device.

9. The stand of claim 8, further comprising:
a plurality of holes that are positioned along each of the pair of mounting plates, and
each of the plurality of holes including a location that is complementary to a location of a threaded mounting aperture on the back surface of the light therapy device.

10. The stand of claim 1, wherein the panel attachment assembly is hingedly connected to the adjustable arm assembly.
